SEQUENCE LISTING (56 sequences)

SEQ ID NO: 1 - DNA, 18 bp, Artificial sequence
  primer
acacctcccgcaaccccc

SEQ ID NO: 2 - DNA, 18 bp, Artificial sequence
  primer
acgcacccatccaccccc

SEQ ID NO: 3 - DNA, 18 bp, Artificial sequence
  primer
ccacccagcatcaccccc

SEQ ID NO: 4 - DNA, 18 bp, Artificial sequence
  primer
gccatcccaccaacctcc

SEQ ID NO: 5 - DNA, 18 bp, Artificial sequence
  primer
ccctcgaacaccacctcc

SEQ ID NO: 6 - DNA, 18 bp, Artificial sequence
  primer
acccgccactcaacctcc

SEQ ID NO: 7 - DNA, 18 bp, Artificial sequence
  primer
cgacaccaacctccgacc

SEQ ID NO: 8 - DNA, 18 bp, Artificial sequence
  primer
cccacaagccactcgacc

SEQ ID NO: 9 - DNA, 18 bp, Artificial sequence
  primer
caaccgactccaccgacc

SEQ ID NO: 10 - DNA, 5 bp, Artificial sequence
  primer
cctcc

SEQ ID NO: 11 - DNA, 23 bp, Artificial Sequence
  Primer
caccaccaccacctctcacctcc

SEQ ID NO: 12 - DNA, 17 bp, Artificial Sequence
  Primer
cctccaacaccacctct

SEQ ID NO: 13 - DNA, 17 bp, Artificial Sequence
  Primer
cctccttcttctcctcc

SEQ ID NO: 14 - DNA, 18 bp, Artificial Sequence
  Primer
ggaggtggtgttcgaggg

SEQ ID NO: 15 - DNA, 17 bp, Artificial Sequence
  Primer
ggaggtggtgttggagg

SEQ ID NO: 16 - DNA, 20 bp, Artificial Sequence
  Primer
caccctccaacaccacctcc

SEQ ID NO: 17 - DNA, 23 bp, Artificial Sequence
  Primer
cctccaacaccaccaccacctcc

SEQ ID NO: 18 - DNA, 23 bp, Artificial Sequence
  Primer
cctccaacaacaacaccacctcc

SEQ ID NO: 19 - DNA, 17 bp, Artificial Sequence
  Primer
ccaccaacaacaccacc

SEQ ID NO: 20 - DNA, 17 bp, Artificial Sequence
  Primer
cctccaacaccacctcc

SEQ ID NO: 21 - DNA, 20 bp, Artificial Sequence
  Primer
cctccaacaacaccacctcc

SEQ ID NO: 22 - DNA, 17 bp, Artificial Sequence
  Primer
cctccaacaccaccacc

SEQ ID NO: 23 - DNA, 18 bp, Artificial Sequence
  Primer
ccctcgaacaccacctcc

SEQ ID NO: 24 - DNA, 23 bp, Artificial Sequence
  Primer
ctccaccaccaccactctccacc

SEQ ID NO: 25 - DNA, 17 bp, Artificial Sequence
  Primer
cctccaacaacaccacc

SEQ ID NO: 26 - DNA, 20 bp, Artificial Sequence
  Primer
cctccaacaccaccacctcc

SEQ ID NO: 27 - DNA, 23 bp, Artificial Sequence
  Primer
tccacctccaccacacctccacc

SEQ ID NO: 28 - DNA, 23 bp, Artificial Sequence
  Primer
ccatccacctccaccacacctcc

SEQ ID NO: 29 - DNA, 17 bp, Artificial Sequence
  Primer
ccaccaacaccacctcc

SEQ ID NO: 30 - DNA, 17 bp, Artificial Sequence
  Primer
ccaccaacaacacctcc

SEQ ID NO: 31 - DNA, 23 bp, Artificial Sequence
  Primer
ctcctccaccaccaccacctacc

SEQ ID NO: 32 - DNA, 23 bp, Artificial Sequence
  Primer
ctccaccctccaacaccacctcc

SEQ ID NO: 33 - DNA, 17 bp, Artificial Sequence
  Primer
cctccaccacaacctcc

SEQ ID NO: 34 - DNA, 17 bp, Artificial Sequence
  Primer
cctccaacaccacctcg

SEQ ID NO: 35 - DNA, 17 bp, Artificial Sequence
  Primer
cctcccaccaaacctcc

SEQ ID NO: 36 - DNA, 17 bp, Artificial Sequence
  Primer
cctccaacaacacctgc

SEQ ID NO: 37 - DNA, 23 bp, Artificial Sequence
  Primer
caccacctctccaccactccacc

SEQ ID NO: 38 - DNA, 17 bp, Artificial Sequence
  Primer
cctccaaaccaccctcc

SEQ ID NO: 39 - DNA, 23 bp, Artificial Sequence
  Primer
ccaccaccacacctcctcctacc

SEQ ID NO: 40 - DNA, 17 bp, Artificial Sequence
  Primer
cctccaacaccagctcc

SEQ ID NO: 41 - DNA, 17 bp, Artificial Sequence
  Primer
cctccaacccaacctcc

SEQ ID NO: 42 - DNA, 17 bp, Artificial Sequence
  Primer
cctccaacaacagctcc

SEQ ID NO: 43 - DNA, 17 bp, Artificial Sequence
  Primer
cctccaacaacacgtcc

SEQ ID NO: 44 - DNA, 17 bp, Artificial Sequence
  Primer
cctccaacaccacgtcc

SEQ ID NO: 45 - DNA, 17 bp, Artificial Sequence
  Primer
cctccaacacccagtcg

SEQ ID NO: 46 - DNA, 17 bp, Artificial Sequence
  Primer
cctccaacaccaccgcc

SEQ ID NO: 47 - DNA, 17 bp, Artificial Sequence
  Primer
cctccaacaccacctgc

SEQ ID NO: 48 - DNA, 17 bp, Artificial Sequence
  Primer
cctccaacaccccactc

SEQ ID NO: 49 - DNA, 17 bp, Artificial Sequence
  Primer
cctcccaacaaccctcc

SEQ ID NO: 50 - DNA, 17 bp, Artificial Sequence
  Primer
cctccaacacccctcac

SEQ ID NO: 51 - DNA, 17 bp, Artificial Sequence
  Primer
cctccaacaccaccttc

SEQ ID NO: 52 - DNA, 17 bp, Artificial Sequence
  Primer
cctccaacaccacctac

SEQ ID NO: 53 - DNA, 23 bp, Artificial Sequence
  Primer
ccacacctccactccacacctcc

SEQ ID NO: 54 - DNA, 17 bp, Artificial Sequence
  Primer
cctccaacaccacctca

SEQ ID NO: 55 - DNA, 17 bp, Artificial Sequence
  Primer
cctccaacaacacctcc

SEQ ID NO: 56 - DNA, 17 bp, Artificial Sequence
  Primer
ccaccttcttctccacc

(19) United States
(12) Patent Application Publication
(10) Pub. No.: US 2006/0105368 A1
(43) Pub. Date: May 18, 2006

(54) Method and device for simultaneously molecularly cloning and polylocus profiling of genomes or genome mixtures

(76) Inventor: Leigh A. Burgoyne, Bedford Park S.A. (AU)

Correspondence Address: Amy E. Rinaldo;KOHN & ASSOCIATES, PLLC, Suite 410, 30500 Northwestern Highway, Farmington Hills, MI, 48334, US

(21) Appl. No.: 11/255,691

(22) Filed: Oct. 21, 2005

Related U.S. Application Data
(63) Continuation-in-part of application No. 10/486,440, filed on Feb. 9, 2004.
(60) Provisional application No. 60/313,912, filed on Aug. 21, 2001.

(51) Int. Cl. C12Q 1/68; C12P 19/34

(52) U.S. Cl. 435/6; 435/91.2

(57) ABSTRACT

A method for amplifying genetic material to characterize complex populations of nucleic acids and a method for applying these characterizations to applications. The characteristics of a population of nucleic acids may be an index of disease states as for example when pathogens cause nucleic acid release into body fluids or they may be for example, an index of soil usage. Although characterization on arrays may be performed conventionally with one defined sequence on each array spot, in the preferred form each spot in the array is not a single sequence but is a sample of sequences representative of a population.

### CROSS-REFERENCE TO RELATED APPLICATIONS

This application is a Continuation-in-Part of U.S. patent application Ser. No. 10/486,440, filed Feb. 9, 2004, which claims the benefit of priority of PCT/US02/26670, filed Aug. 21, 2002, which claims the benefit of priority under 35 U.S.C. Section 119(e) of U.S. Provisional Patent Application No. 60/313,912, filed Aug. 21, 2001, which is incorporated herein by reference.

### BACKGROUND OF THE INVENTION

1. Field of the Invention

The present invention generally relates to the fields of molecular biology and nucleic acid analysis. More specifically, the present invention relates to a method of genetic analysis designed for characterizing complex mixtures of nucleic acids.

2. Description of the Related Art

Molecular cloning is the process of selecting a nucleic acid sequence and amplifying that sequence many times. Profiling is normally understood to be the process of selecting a small subset of sequences out of a genome; one at the minimum. Profiling as it relates to the present invention means the development of a characteristic sequence or set of sequences from within a larger complex pool of sequences, such that the characteristic set of sequences have utility (e.g. disease diagnostics, criminal identification, quality control and population genetics).

These processes are used for detection and identification of diseases, organisms and/or their effects that are either novel to the world, or are previously described organisms whose presence are unsuspected in a serum or plasma. Detection and identification of novel pathogens are currently accomplished by formally detecting the genome of the pathogen with specific sequences from known regions of the genome using methods such as those disclosed in U.S. Pat. No. 6,255,467.

Microbial identification, i.e. identification of bacterial, viral, and mycotic species, strains, and subtypes, is a key concern in clinical microbiology for diagnosis of infectious disease, selection of effective pharmaceutical treatment, and epidemiological investigation of the source and spread of infectious disease. Microbial identification is also a vital requirement in the detection and management of biological warfare agents. Microbial identification is important in agricultural, industrial, and environmental biomonitoring. For example, microbial identification can be used for the detection of pathogens that reduce agricultural productivity as well as for microbes that add nutrients to soil, in order to monitor industrial bioprocesses and assess biodegradation capacity in soil and waste treatment facilities. This technology works best for when nucleic acid templates are present at “trace” levels.

Analysis of gene expression is another area that requires new methodologies in order to function more effectively. Transcriptional profiling, i.e., analysis of the relative abundance of messenger RNA (mRNA) transcribed from different genes, is critical to understanding patterns of gene expression that are associated with all biological processes including development, differentiation, response to environmental stresses, and other cellular and organism-based functions of interest. The ability to analyze patterns of gene expression can lead to discovery of new genes associated with biological processes. A detailed understanding of gene regulation at the level of transcription is also a significant concern of the pharmaceutical industry. The understanding of gene regulation enables the identification of genetic targets for drug development that can lead to understanding the heterogeneous responses to pharmaceutical interventions. Transcriptional profiling is currently conducted by the techniques of “differential display” (Liang, P. and Pardee, A. B. (1992) Science 257:967-971; Liang, et al., (1994) Nucl. Acids Res. 22:5763-5764; Prashar, Y. and Weissman, S. M. (1996) Proc. Nat'l. Acad. Sci., U.S.A. 93:659-663.) and “representational difference analysis” (Hubank, M. and Schatz, D. G. (1994) Nucl. Acids Res. 22:5640-5648; Lisitsyn, N. A. (1995) Trends Genet. 11:303-307), both of which involve PCR, gel electrophoretic analysis of DNA fragments, and a variety of other complex manipulations. A need clearly exists for new technology, an alternative to DNA arrays that enables more robust, rapid, and cost-efficient methods for analyzing a very large number of gene transcripts in a short period of time.

An example of such a method used in the field of detection is random amplified polymorphic DNA (RAPD) marker analysis. The method utilizes a single, short primer PCR with genomic DNA. The single, short (8-10 mer) primers have an arbitrary sequence and generate a product that can be used in gel electrophoretic fingerprint analysis to generate numerous polymorphic markers (Williams et al, (1993) Methods in Enzymol. 218: 704-740; McClelland & Welsh, (1995) pg 203-211. In: Dieffenbach, C. W., Dveksler, G. S. (Eds.) PCR Primer—A Laboratory Manual. Cold Spring Harbor Laboratory Press, USA.).

Additionally, amplified fragment length polymorphism (AFLP) uses a single primer to profile nucleic acids. The primer is ligated to restricted fragments and has different principles of amplification to those disclosed herein (Vols et al, (1995) Nucl. Acids Res. 23(21): 4407-4414; Gibson et al, (1998) J Clin Micro. 36(9):2580-2585).

Microbial identification typically involves time-consuming and expensive culturing and biochemical procedures, as well as costly and complex immunological tests. DNA sequencing and PCR analysis also can be performed to achieve accurate microbial identification and typing, however, these microbial DNA diagnostic tests require pre-knowledge of the sequences expected to be found so that PCR primers can be designed, or sequences prepared, for use as array points. When the sequences are used as point arrays, classic identification studies need to be performed, often by culturing the organism followed by classic sequencing of the genome. Additionally direct pathogen identification is usually only possible when host or contaminating nucleic acid concentrations are low.

The identification of highly divergent or novel genomes is typically performed by inserting DNA into a vector such as a plasmid or a virus and then selecting clones randomly and sequencing the inserts. This is time-consuming, usually requiring weeks to be completed. Additionally, the method requires a relatively large amount of the nucleic acid. Because of these limitations, the method is almost never used in routine diagnostics.

Although not suitable for routine diagnostics, the cloning and sequencing procedure of traditional molecular biology does provide sequences sampled from the subject genome that can be observed and fully analyzed, even though the sequences have never been previously seen. A limitation of this approach is that culturing unknown organisms to acquire sufficient nucleic acid to perform the procedure is unreliable.

In summary, most current technologies for the “rapid” identification of sequences requires precise information about a sequence, or identification of the sequence following a long and tedious procedure. Thus, the presence of a pathogen of completely unknown sequence is not routinely detectable because identification by PCR requires known, stable regions of the candidate genome to act as sources for the primer sequences. Also, the identification of sequences on a sequence-array requires a very large menu of candidate sequences to be physically attached to that chip at individual spots. Therefore, neither of these techniques is suitable for highly divergent or novel genomes.

It would therefore be useful to develop a method that does not require culturing of an organism to obtain conventionally useable amounts of DNA, and that is much faster and more readily automatable than the methods disclosed above. It also would be useful if this method could deliver an output of sequences that can be analyzed against the world's databases for match or non-match and can be analyzed for relationships, even distant relationships, to known sequences.

It would also be useful to develop an effective method for rapidly sampling and sequencing molecularly cloned samples. These can be used for rapid identification of the amplified nucleic acids, or if novel, transcriptional profiling or classification of species, strains, and sub-types.

### SUMMARY OF THE INVENTION

According to the present invention, there is provided a method and examples for amplifying genetic material by selecting sequences based on the properties of the sequence found between primer binding sites. There is also provided a kit for performing the above method including a list of primer sequences and a device for amplifying genetic material. Finally, there is provided an example of a computer program for creating the primers for use in the above methods.

### BRIEF DESCRIPTION OF THE DRAWINGS

Other advantages of the present invention will be readily appreciated as the same becomes better understood by reference to the following detailed description when considered in connection with the accompanying drawings wherein:

FIG. 1 is a graph showing the methylene blue photolysis effect versus archival time of human DNA on filter paper;

FIGS. 2A through D are photographs and graphs showing the results of amplification of genetic material using the SMIPS primer method of the present invention;

FIGS. 3A through D are photographs and graphs showing the results of amplification of genetic material using the SMIPS primer method of the present invention, FIG. 3D shows the amplification protocol;

FIG. 4 is a photograph showing the results of amplification of DNA using four stable DNA polymerases;

FIGS. 5A and B are photographs showing in FIG. 5A an agarose gel stained with ethidium bromide and in FIG. 5B x-ray film exposed to Southern blot hybridized with ³⁵S dATP labeled soil amplification;

FIG. 6 shows a block diagram of the devices of the present invention;

FIG. 7 is a diagram showing the four major structural formats observed in SMIPS amplification;

FIG. 8 is a bar graph depicting primers that perform as equivalents to different degrees; and

FIG. 9 is a bar graph depicting cross-reaction of SMIPS amplification products that originated from soil samples obtained from a variety of locations.

### DESCRIPTION OF THE INVENTION

Generally, the present invention provides a method and kit for amplifying RNA and/or DNA in a sample while simultaneously producing molecular clones that also constitute a profile of the sample. The method can be used for the analysis of patient wellness, detecting illness as well as determining the presence of bacteria or other pathogens. It should be noted that spectra of sequences that act as indices of “wellness” or “illness” do not necessarily belong to the pathogen causing the main symptoms of the disease or even to any other pathogen. The method also can be used for agricultural purposes such as testing for nucleic acids in soil samples or other similar purposes.

A “profile” as used herein is a characteristic set of sequences within a large number of sequences such that the characteristic sequences have some utility (e.g. criminal detection, disease diagnostics, quality control, population genetics, scientific research). A particular feature of the invention is that the profiles are easily amplified.

The term “product” as used herein is used to indicate a mixture as opposed to a single sequence. In other words, the term “product” can be used interchangeably with the term “products” as the product being referred to is a mixture of sequences and not a single sequence.

A “complex template” is a template made of an effectively infinitely complex mixture of sequences such that there is no significant repetition of sequences except as occur by coincidence. An example of a complex template is the mixture of sequences that are obtained from an agricultural soil typically composed of many microbial genomes in highly disparate amounts. Thus, the complex template includes a large variety of sequences that can be present in varying quantities.

A “non-complex template” refers to a simple mixture of sequences. An example of a non-complex template is a single bacteriophage genome. An intermediate example is the human genome, which contains a large amount of repetitive DNA as well as a large number of sequences that are present in only two copies.

The “degree of selective power” of a primer is defined as the proportion of a complex template that the primer amplifies during the relevant amplification. Early in amplification, selection is dominated by the tertiary structure of the template and its effect on primer interactions during annealing stages of amplification. Subsequently, selection becomes dominated by the overall template properties of the completing products.

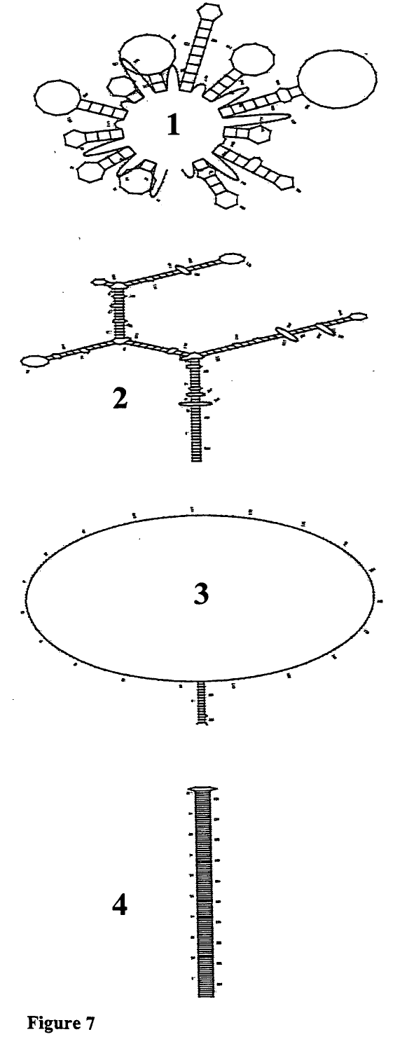

The term “SMIPS” refers to a fundamental principle of the invention, structurally mediated interprimer selectivity (SMIPS). “SMIPS amplification” is defined as an amplification utilizing at least one SMIPS primer. The SMIPS amplification combines a SMIPS primer with conditions of the amplification process that favor the degree of selective effect desired. The SMIPS amplification is distinct from arbitrary primed PCR in that the selection principles of SMIPS function in relation to the types of tertiary structures a given sequence forms during the annealing stages of the amplification reaction. This type of selection is Darwinian in nature and proceeds until there is a small population of successfully amplifying species. Most other types of amplification processes, including Arbitrary Primed PCR [AP-PCR] try to avoid this type of selection. Certain tertiary structures provide greater accessibility for primer loading than others (for example, FIG. 7, Format 1). The accessibility for primer loading provides the main mechanism of selection.

A “SMIPS primer” is defined as a primer capable of amplifying sequences of nucleic acids. Examples of such primers are listed throughout the present application and in Table 3. Further, equivalents and non-equivalents of the primers listed in Table 3 can also be utilized in conjunction with the SMIPS amplification. The only requirement of the SMIPS primer is that the primer has the ability to function in the amplification process as defined herein.

A “primer/primer comparison” is defined as the binding of the product from an amplification of a complex template to a spot of product from the same template amplified by a different primer. In a primer/primer comparison, a cross reaction of more than 5% of the mixture of products of one primer with another, allowing for blanks and non-equivalent controls, make the primers “equivalent”. A 5% cross-reaction is considered the threshold. In other words, 5% cross-reaction is the highest (i.e. least stringent) of the conventionally accepted level of cross-reaction that can be allowed. Whether a primer is being used as an equivalent is a function of both the primer sequence and the amplification conditions.

“Equivalent primers” are primers that have equivalent spectra of products. Equivalent spectra of products are products with at least 5% overlap of homology (5% was chosen as the least stringent, conventionally accepted limit of significance in conformity with P=0.05 in conventional statistics). Non-equivalents product spectra, which are selected out of the same complex template, are defined as sub-classes of sequences that do not significantly overlap.

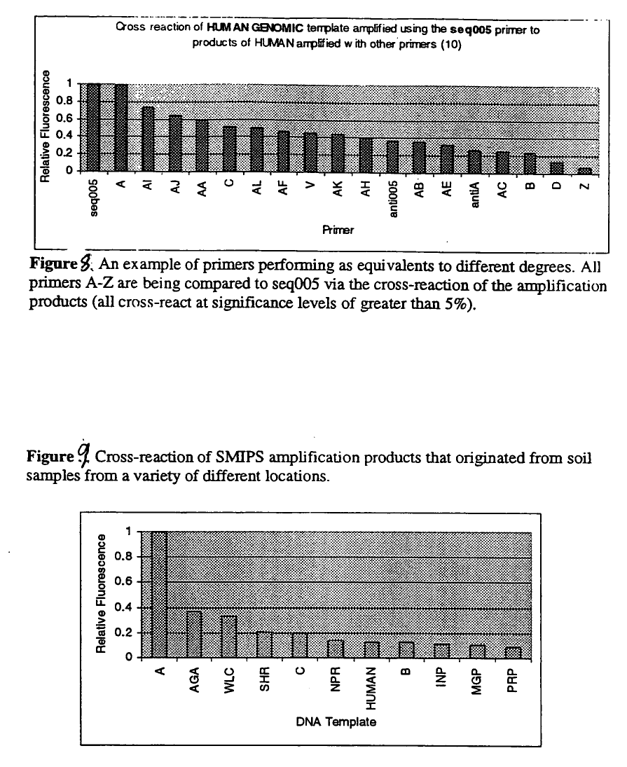

The term “equivalence” for either primer or products is defined by the cross reaction that occurs when the template is a complex template. In extremely simple mixtures of sequences, such as those of a single viral genome, cross-reaction could occur by simple chance. This type of cross-reaction referred to, as “non-equivalent cross-reaction” is not defined as equivalence. An example of a non-equivalent cross-reaction is found in the human genome. The human genome has a great deal of repetition, so any repetitive elements selected by chance will have a significant degree of cross-reaction by chance. A specific example demonstrating this is found in FIG. 8, in which a cross-reaction is observed between product from seq005 and product from anti005 (anti005 primer having the exact complementary sequence to seq005, and thus should initiate at very similar sites but amplify in the opposite directions). It is highly unlikely that these primers would amplify sequence regions with the level of cross-reaction observed except to the degree to which the cross-reaction are occurring because of highly repetitive sequences in the template.

“SMIPS cross-reactions” are defined as cross reactions that occur between greater than 5% of the two reaction mixtures. The cross reactions arise from two sets of products cross-reacting with one another, wherein at least one product set is derived from a complex template. Only primers that are “equivalents” create SMIPS cross-reactions.

“Simple-sequence cross-reactions” are defined as the cross-reactions of the type generated by fortuitous amplifications of overlapping sequences from extremely simple templates. Any primers at all, used in any pair-wise combinations, can create simple-sequence cross-reactions.

A true “blank” is defined as the binding of the fluorescent products of amplification from one template mixture to the products from a completely unrelated set of complex sequences. At least one template mixture must be highly complex for the blank to apply. An example of a blank are the products amplified from soil DNA binding to a spot of amplification product from the human genome.

Primer loading sites are sites that primers initiate from and may have no homology to the primer, or complete homology to the primer, or anything in between.

The SMIPS primers and conditions have a variety of applications, including forensic analysis, pathological diagnostics, population genetics, and quality control. Applications of SMIPS primers and the conditions used to analyze and match complex mixtures of sequences can be accomplished without isolation of, or even consideration of the organisms that make up the individual components of that mixture. The method of the present invention can provide a repeatable selection of sequences, when applied to a nucleic acid mixture. SMIPS primers and conditions can be applied to any situation in which levels of cross-reactivity can be compared between amplification products and/or other nucleic acids of choice, and analyzed using a variety of technologies, including DNA array or chip analysis and southern blotting. The analysis can use both SMIPS cross-reactions and simple sequence cross reactions together or apart.

The information gained from SMIPS can be an empirical measure of the degree of sequence sharing, the overall similarity, of two extremely complex mixtures of sequences. The information gained from simple sequence cross-reactions can provide an empirical measure of the simplicity of any one mixture of sequences. Thus, the use of the primer-list in the manner described allows useful information to be gained about populations of sequences rather than individual sequences or organisms that contribute to the mixture.

In patient wellness applications, the present invention functions by analysis of the amounts, types and ratios of nucleic acids present in plasma or blood lysates. The genetic content of the amplifications is diagnostic of types and stages of disease, irrespective of whether the pathogens sequences are amplified or not.

In detecting illness, the present invention functions by detecting diseases based upon changes in the amounts of nucleic acids that are present in plasma or serum, agricultural samples, soil, or other samples. Examples of diseases that can be detected include infectious bacterial and viral diseases.

An example of an agricultural or forensic application includes, but is not limited to, biologically profiling soils from minute samples of soil. The genetic content of a sample of soil is extremely variable and can be highly complex. Soil samples can contain genetic material from a variety of sources, such as, soil bacteria and fungi, and cellular debris from other organisms (e.g. skin or blood from passing animals). Microbes found in soils can vary considerably from location to location depending on the local environment (e.g. moist or dry, agricultural or forest). Analyses of the genetic materials found in soil samples have revealed data that have applications in both agriculture and forensic diagnostics. For example, the range of sequences matched in a particular soil sample to another soil sample by indicating similarity between the soil's sequences can provide an indication as to the type of crop that would provide the highest yield. Alternatively, in forensic diagnostics, the genetic make-up of a soil sample taken from a piece of clothing compared to that taken from a crime scene could provide evidence to link a suspect to a crime. In agricultural applications, the present invention can also function by detecting foreign nucleic acids that should not be found in the sample.

The method and kit of the present invention differs from many of those found in the prior art, e.g., AFLP, RAPD, or conventional two primer PCR, because the prior art methods and kits for amplifying RNA and/or DNA often result in what are known to those of skill in the art as “primer-dimers” and other primer concatenates. Primer concatenates are a range of short to very long products of the primers formed by template switching of polymerases of which the smallest member is a primer-dimer. These artifacts are artificial by-products of the PCR and reverse transcriptions and they quench amplification. Primer-dimers and primer concatenates generally are undesirable by-products of polymerase reactions. Therefore, the present invention is beneficial over the amplification reactions of the prior art because the present invention is extremely resistant to the formation of primer-dimers and primer concatenates.

The present invention is able to overcome the problems of the prior art by utilizing primers that bind efficiently to nucleic acids, but have minimal specificity for defined target sequences. The primers also do not readily associate in such a way as to allow amplifiable concatenations, even at low annealing temperatures. Typically, these primers are approximately 16-30 bases long, and have properties as described below.

The prior art discloses the use of primers that either require the use of a ligase or sequence specificity for the sequence to be amplified. Unlike the prior art patents, the present invention does not require the use of a ligase nor does it require significant sequence specificity from the primers. By using suitable primer sequences and long extension times, the PCR can be used to sample a low concentration of nucleic acid molecules in a sample without assuming or knowing anything about the sequences included therein. A long extension time can be defined as a time long enough for at least 2 kb of extension by the polymerase in the amplification mixture. If many amplification cycles are used, then the method of the present invention also enables the investigator to profile the sequences found in the sample such that the profile becomes progressively clearer and simpler with an increased number of amplification steps. Additionally, the present invention does not require large amounts of nucleic acid, as are commonly required by conventional amplifications, in order for amplification to take place. For example plasma or serum from a healthy person contains very little nucleic acid but a sample as little as 1 ul can be analyzed by the present invention to provide comparisons with similar samples from patients for illness determination (e.g. by automated processing).

By utilizing suitable, primer sequences and short extension times, PCR also can be used to sample a very small amount of nucleic acid molecules without assuming or knowing anything about the sequences included therein. A short extension time is defined as the time it takes for the PCR system to produce approximately 200 base-pair extensions in that particular amplification mixture. The PCR amplifies DNA or RNA post reverse transcriptase. If RNA is to be amplified, the PCR is preceded by reverse transcriptase copying of the RNA to DNA, using the same single template as is used in the PCR that follows the copying. If single-stranded species, either RNA or DNA, is to be selectively favored in the amplification in the first extension cycle, either reverse transcriptase or PCR is not initiated by, nor preceded by, a denaturation step.

Many amplification cycles imposed on the very small DNA samples disclosed above enable the method to become highly selective in the amplification of sequences. This selectivity does not arise from the same principle as does the selectivity of conventional PCR. The method of the present invention instead selects particular sub-sequences of the interprimer sequence to amplify in a process termed structurally mediated interprimer selectivity. The selection is based upon the properties of the amplified section instead of the complimentarity of primers for target sequences as is found in conventional PCR. Importantly, the method creates a relative selectivity for long sequences. Additionally, the amplifications avoid, or at least minimize, any target sequence specificity for primers. The SMIPS process is accomplished when primers are first annealed for one or two cycles at very low specificity so that there is little or no sequence specificity. This step is then followed by a series of amplifications that use a higher specificity of amplification such that there is no more initiation from the original template. The products of the initial amplification that preferentially amplify are those with interprimer sequences highly favorable to amplification. Therefore, the method preferentially amplifies sample sequences from meta-genomes, which are defined as a complex mixture of one or more genomes, e.g., a mixture of the human, mitochondria and one or more parasitic genomes, or more complex genomes than from very simple genomes because the more complex a genome, the higher the probability of unusually amplifiable interprimer sequences are contained therein.

SMIPS primers are used to amplify nucleic acid sequences. The primer sequences become incorporated into sections of product at the 5′ and 3′ ends. Competition then exists between the sections of amplifying template favoring those nucleic acid molecules that least hinder primer-loading interactions. This steric hindrance is a function of the composition of the nucleic acid sequences amplified between the SMIPS-primer-homologous ends. For example, highly competitive sequences for primer interaction are those with termini uninvolved in tertiary structure formation enabling primer-loading and which may thus be amplified in preference to other nucleic acid sequences that have less accessible primer-loading sites. (Primer loading sites are sites that primers initiate from and may have no homology to the primer, or complete homology to the primer, or anything in between.)

The SMIPS amplification process can be manipulated to allow different sequences to form the preferred structures and therefore be selected for amplification. Primers facilitate the selection based upon the primer's nucleotide content compared with the content of the template, along with the second-order structures formed by the template (examples shown in FIG. 7). Also, unlike arbitrary primed PCR, the products of SMIPS amplification are commonly assessed by sequence-dependent technology that is insensitive to the lengths between initiation sites rather than the size of the amplified bands.

Using SMIPS primers and SMIPS amplification protocols versus a pair of conventional primers and protocols, stops the selectivity of the PCR from being dominated by the homology of the primers to the template (the traditional selectivity of the PCR) and instead makes selectivity dependent on the properties of the amplified section between the primers.

Amplification favors interprimer sequences that, when single-stranded, fold such hat their termini are held well apart. This is a strange property and is only well developed by relatively few sequences, even in a very complex genome, and is often not found in a simple genome. Further, the section of sequence between the primers, commonly has a high tendency to fold in such a way as to positively oppose the proximity of its ends. The uninvolvement of termini in the tertiary structure enables primer loading and constitutes the principle reason for the primer selectivity disclosed herein and hence the ability to create profiles.

The structural conformation most favored by SMIPS amplifications is of the type seen in format 1 (FIG. 7). The conformation forms in such a way to keep the ends of the structure clear for primer loading. The type of structure in format 3 (FIG. 7) can also be utilized if the interprimer sequence is of significant size. Structural conformations identified of the type observed in formats 2 & 4 are selected against, as they do not promote efficient primer loading (e.g. the ends of the template are occupied). More specifically, specific folding formats include: CFS formats (Completely Folded Sequence, format 2, FIG. 7) are complex folding patterns that positively block primer loading; hair-pin loop type structures (when only a single primer is used) (HPS, format #3, FIG. 7) favors long inserts that disrupt the structure and preferentially inhibit the formation of concatenation artifacts of PCR that arise from repetitive copies of the primer, as these favor closure, HPS structures favor interprimer sequences that prevent the 5′ and 3′ ends from coming together by the entropic effects of a long insert (from thermal writhing); and SLS formats (Stem Loop Sequences, format #4, FIG. 7) are simple hairpins that should very strongly and positively block primer loading.

By using combined molecular cloning and profiling (sample-amplifying), pathogenic genomes that are plasma or serum-loaded or are from other sources such as soils, then can be estimated by DNA-array-related technology or by scanning electrophoretograms or, as in this disclosure, by absolute sequencing. In traditional PCR, one primer pair gives one product so a profile requires multiple primers in a multiplex. In profiling using SMIPS primers, SMIPS primers give many products that have similar amplification properties characteristic of the section between the primers.

The principle of profiling complex genomes, such as that of humans as understood in the forensic and related sciences, is the selection of a few alleles for amplification that are peculiarly advantageous for particular applications. The present invention utilizes the SMIPS primers long-extension reaction with many cycles to naturally accomplish this principle by selecting for strange, single-stranded structures. The application of this principle creates a base-process for automated molecular cloning and repeatable sampling and sequencing from unknown genomes.

The method of the present invention is a very high amplification-PCR reaction with primers that can be preceded by reverse transcription with the same primers. SMIPS primers of 16 to 30 bases long are used at low stringency, such that zero homology or less than 6 to 8 bases of the template have homology to the primers' 3′ end that serves as an initiation site. The rest of the primer serves to inhibit amplification of primer-dimers and primer concatenates.

More specifically, the amplification first amplifies many sites on the template, but then progressively favors an ever-smaller number of sites so that a limited number of multiple products from a complex genome slowly resolve into progressively simple banding patterns. The products thus evolve during the amplification from evenly polydispersed distributions of size to a progressively simpler subset of amplification products that are characteristic of the original template. These products comprise a profile in that they are a complex mixture of products that are characteristic or diagnostic of the genome or genomes from which they originated.

When many rounds of amplifications are performed, the process becomes strongly selective for the properties of the sequences between the primers. Such properties can include, but are not limited to a single stranded nucleic acid under the prevailing temperatures of its primer-loading stage, and in amplification, folds so as to most effectively allow primer loading. This is only one specific condition/property; so the larger a genome, the higher the numbers of sequences within the genome that fulfill this condition. A small genome only amplifies well if it possesses a sequence that folds appropriately when single stranded, or is amplifying in the absence of more complex genomes. Therefore, the process statistically samples from large genomes.

By using the same primers to prime the reverse transcriptase as is used for the PCR, favoritism is obtained that preferentially amplifies RNA sequences over DNA sequences. Another effect is that when the RNA is primed non-specifically with the SMIPS primers, the copy of the RNA, with the SMIPS primer now grafted onto it, preferentially primes its complementary genes in the contaminating DNA such that the DNA 5′ to the original RNA tends to be preferentially entrained in the subsequent PCR.

The selection of parasitic genomes maybe enhanced by selectivity for single-stranded genomes due to not denaturing the template before the first extension because this allows preferential priming of single-stranded nucleic acid over double-stranded nucleic acid.

The present invention can be also used to amplify nucleic acid from a parasite in the plasma or serum and can be made to favor RNA sequences. The methods and products of the present invention can be used to detect known or unknown bacteria, either RNA or DNA, with equal speed and ease, example 1 & 3 illustrate this utility. The method can also be used to detect virions.

Combining the ability to detect illness and determine patient wellness as disclosed in the previous embodiment with the ability to identify pathogens at low concentration, even if indirectly, provides broadly based objective evidence of illness for legal or industrial purposes. This differs from the prior art applications of PCR or RT-PCR in association with the detection of a parasitic organism in that the methods of the present application are applicable to the association of the presence of one or more organisms as well as evidence of tissue damage whereas the prior art methods are not applicable to said association.

The ability to monitor the progression of disease from the sequence drift of the pathogen is beneficial in the fight against infections. With regard to HIV patients, the detection of secondary pathogens, directly or indirectly, in the serum or plasma of a patient enables investigators to determine much about the state of the disease and its prognosis. Analysis of the ratio of host genetic material (e.g. genomic to mitochondrial or RNA messages) can offer insights into disease progress and patient health.

The present invention has wide applications, for example, for use in field studies of microbes or in general practice surgeries for routine diagnostics. The present invention is automatable and as such has utility in mass screenings and in the detection of previously undetectable infective moieties.

The invention is particularly applicable to systems that handle the acquisition and analysis of complex data in databases that associate clinical records with molecular data. Analysis of the amplification products by various means is common to the nucleic acid field. Examples of such amplification products include, but are not limited to, a “DNA chip,” high resolution gels with data acquisition systems, post-chip technology, on-line sequencing technology, or any other suitable technology known to those of skill in the art.

The methods of the present invention can be also used in conjunction with a material that can store genetic material. This material can be beneficial because it is amenable to distance-collection and is highly automatable with an extremely useful and very broad application. This methods-material combination combines the following:
Processing for RNA occurring on the storage media.
Low specificity, high-gain amplification using very few primers and, in the preferred and demonstrated version, long-range PCR. (Importantly, not random primers.)
The degree of specificity can be optimized for general use from the choice of amplification conditions, generalized to the choice of the amplification parameters.
Final data analysis by nucleic acid arrays or on-line sequencing technology.

The present invention also allows open-ended accumulation of sequence libraries for use on chip-style devices.

The present invention can be used for measuring the frequency, occurrence and levels of nucleic acids. The methods measure levels of nucleic acids more sensitively than current technology, without respect to a specific organism. In other words, the scope of organisms includes all organisms with nucleic acid, including but not limited to, virions or bacteria in plasma or serum. The methods do have a statistical selectivity for the more complex genomes. The methods therefore only leave out non-nucleic-acid-infective entities such as true prions.

The methods can be also used to objectively record and catalogue large numbers of previously unidentified organisms as gel patterns for future reference. Typing complex mixtures of organisms can be also accomplished by the methods of the present invention. This includes characterizing the nucleic acids from soils for forensic purposes.

The theory of primer design is that they are to have a high C (cytosine) content with very low G (guanine) content, such that their double-stranded products have a high melting point with the primer itself having negligible secondary structure. Purines, particularly G, are to be avoided in the five nucleotides at the 3′ terminus. This is designed to create amplification products with short, high melting point termini, created by the primer, that carry lower melting point nucleic acid sequences; or for sequences that have the property of folding so as to hold the termini apart; or for sequences with a combination of these two properties.

Shown in Table 3 are primers that fulfill these conditions as well as with examples of primers that do not. The primers used in accordance with the above methods do not deliberately prime specific nucleotide sequences. What homologies exist are fortuitous and undesirable. This list is included for purposes of illustration and is not intended to be limiting.

Suitable primers (notably those with the CCTCC 3′ end) were generated using a program that randomly generated a particular sequence-type. Sequences rich in C but low in G, combine a relatively high melting point with negligible self-complementarity.

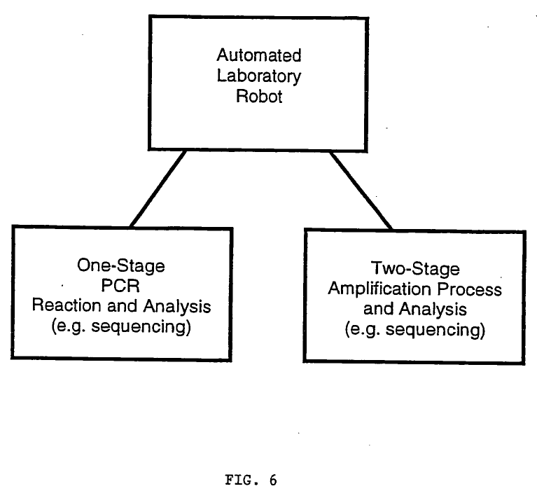

With regard to the device that can be used in conjunction with the above methods, there are at least three mechanization options, including the devices disclosed below. All three options begin with a conventional PCR processing laboratory robot that carries out either a one-stage PCR reaction (devices A and C), or a two-stage amplification (device B). The device is generally shown in FIG. 6.

Device A is a nucleic acid profiling machine. This machine resembles a laboratory robot that can handle 96 well plates that are set beside a capillary electrophoresis machine. The laboratory robot is for two-stage PCR that has a minor difference from conventional processing. The robot is attached to a standard capillary electrophoresis device or HPLC device for separating and observing DNA bands, but not necessarily gathering data from the patterns. The device separates the bands or clones and delivers them to an on-line sequencing device such as a mass spectroscope or by the Sanger procedure that delivers the peaks one by one to a multicapillary sequencing device. The first device must either operate so as to separate the strands of the bands being studied, or in the technically simplest version, the preferred version, must operate on bands that are cut by a restriction endonuclease before loading and then separated as single strands. The endonuclease cutting ensures that the fragments are small enough to separate well with current technology and also ensures that the fragments have only one end either probe-labeled or complementary to the single primer that can be required for the sequencing technology of device B or device C. Sequencing can be also performed by the use of a second low-specificity primer loading, with an alternate primer, thus providing each strand with a separate primer for sequencing.

Device B is to be used in line with and subsequent to device A. Device B takes a short, single strand of DNA and sequences it by Sanger sequencing fluorescent primer chain terminating technology or related technology. The restricted band from the gel is ejected into a reaction vessel such as one well of a 96-well plate. A robot then adds a sequencing mixture containing a primer with the same sequence as the single primer used for the original amplification. After sufficient reaction time as is required to develop the spectrum of terminated sequences characteristic of this technology, the mixture is loaded onto a resolving apparatus, for example, a high resolution capillary gel. The sequence data is acquired automatically and used as desired.

Device C is to be used in line with and subsequent to device A. This also takes a short, single strand of DNA and sequences it. In this case, the sequencing is accomplished by mass spectroscopic (MS) technology. In one example of MS technology, after the small double-stranded band is exposed to a second primer such that a short series of diprimer amplifications can occur to allow ejection from the stage-A separation, one strand of the double-strand fragment to be selected using a reporter-group or handle such as biotin is incorporated into the 5′ end of one of the two primers to separate the strands so that only one strand is presented to the MS apparatus and subsequently sequenced. In another version, the strand separation occurs on the first-stage of separation, stage A, by using separation conditions such that denatured and partly refolded single strands are separated from each other by the properties of the folding structure they form. In another version of this apparatus, the sequence of the single primer or its complement is used to separate the strands by preferentially binding or delaying the movement of one strand before presenting them to the MS apparatus. In all cases, the resultant sequence data is collected and used as desired. In an additional version of this apparatus, prior to sequencing, a second low stringency primer loading step is carried out using an alternate sequencing primer. The purpose of this is to introduce a second primer that is different from the first primer, to allow separate sequencing of each strand.

In an additional version of this apparatus, sequencing is carried out with sequencing primers that are composed of the first amplification primer with an extension of 2 or more bases, giving rise to a set of 16 or more sequencing primers depending on the size of the extension (e.g. number of additional bases). A software algorithm is used to align sequences against a database of known sequences to determine their origin. In addition the software algorithm also analyses confounding or ambiguous sequences by attempting to align small regions of a sequence (e.g. 10 bp) at a time and determining the probability of a match. When enough regions have been aligned an overall identification is then made. Confounding or ambiguous sequences that are identified are then added to the database to aid in future analysis.

The general method requires the following steps. First, samples are collected. The samples can be maintained on storage media. Second, the samples are processed via phenolic or other methods with similar purification effect. Third, the sample is reverse transcribed with the protocol set forth in Table 1. Fourth, the PCR protocol set forth in Table 2 is followed. RT-PCR products are ammonium acetate and alcohol precipitated prior to visualization and then run on an acrylamide gel. Fifth and finally, analysis of the amplification products is accomplished using any of the above-described methods.

TABLE 1
Reverse transcription protocol used to generate cDNA

| Temperature | Time |
|---|---|
| 42° C. | 40 minutes |
| 45° C. | 5 minutes |
| 50° C. | 5 minutes |
| 55° C. | 5 minutes |
| 60° C. | 5 minutes |
| 95° C. | 5 minutes |

TABLE 2
PCR protocol used in the first and second round amplifications

| Temperature | Time | Cycles |
|---|---|---|
| 94° C. | 10 minutes | 1 |
| 60° C. | 30 seconds | 40 |
| 55° C. | 30 seconds | 40 |
| 50° C. | 30 seconds | 40 |
| 72° C. | 2 minutes | 40 |

The above discussion provides a factual basis for the use, the methods, and the kits of the present invention. The method used with, and the utility of, the present invention can be shown by the following non-limiting examples.

### EXAMPLES

Methods:

General methods in molecular biology: Standard molecular biology techniques known in the art and not specifically described were generally followed as in Sambrook et al., Molecular Cloning: A Laboratory Manual, Cold Spring Harbor Laboratory Press, New York (1989), and in Ausubel et al., Current Protocols in Molecular Biology, John Wiley and Sons, Baltimore, Md. (1989) and in Perbal, A Practical Guide to Molecular Cloning, John Wiley & Sons, New York (1988), and in Watson et al., Recombinant DNA, Scientific American Books, New York, and in Birren et al. (eds) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659 and 5,272,057; and incorporated herein by reference. Polymerase chain reaction (PCR) was carried out generally as in PCR Protocols: A Guide To Methods And Applications, Academic Press, San Diego, Calif. (1990). In-situ (In-cell) PCR in combination with flow cytometry can be used for detection of cells containing specific DNA and mRNA sequences (Testoni et al, 1996, Blood 87:3822.).

General methods in immunology: Standard methods in immunology known in the art and not specifically described are generally followed as in Stites et al. (eds), Basic and Clinical Immunology (8th Edition), Appleton & Lange, Norwalk, Conn. (1994) and Mishell and Shiigi (eds), Selected Methods in Cellular Immunology, W. H. Freeman and Co., New York (1980).

### Example 1

### Analysis of Infectious Disease State/Nucleic Acid Estimation

Serological clinical samples of patients carrying hepatitis C (HCV), and human immunodeficiency virus (HIV), as well as from healthy patients were cleaned and amplified on FTA® paper (Whatman House, UK, Whatman plc, Whatman International Ltd., Whatman House St. Leonard's Road 20/20 Maidstone Kent ME16 0LS). However, other methods of viral manipulation can be also used. 1 mm discs loaded with clean sera, HCV, or HIV infected sera were routinely used in analysis.

All samples were phenol processed prior to reverse transcription (RT) and PCR, with the same primer, Seq005, used in both sets of reactions. The RT temperature protocol was as follows: 42° C. for 40 minutes, 45° C. for 5 minutes, 50° C. for 5 minutes, 55° C. for 5 minutes, 60° C. for 5 minutes, 94° C. for 5 minutes. The entire exhausted RT reaction was routinely used as the template for the PCR. The PCR temperature cycling protocol was as follows: 94° C. for 10 minutes, 94° C. for 10 seconds, 60° C. for 30 seconds, 55° C. for 30 seconds, 50° C. for 30 seconds, 72° C. for 2 minutes; for 35 cycles. A sec round of PCR with a protocol identical to the first round was often done, using 1 μl of the first round amplification as template.

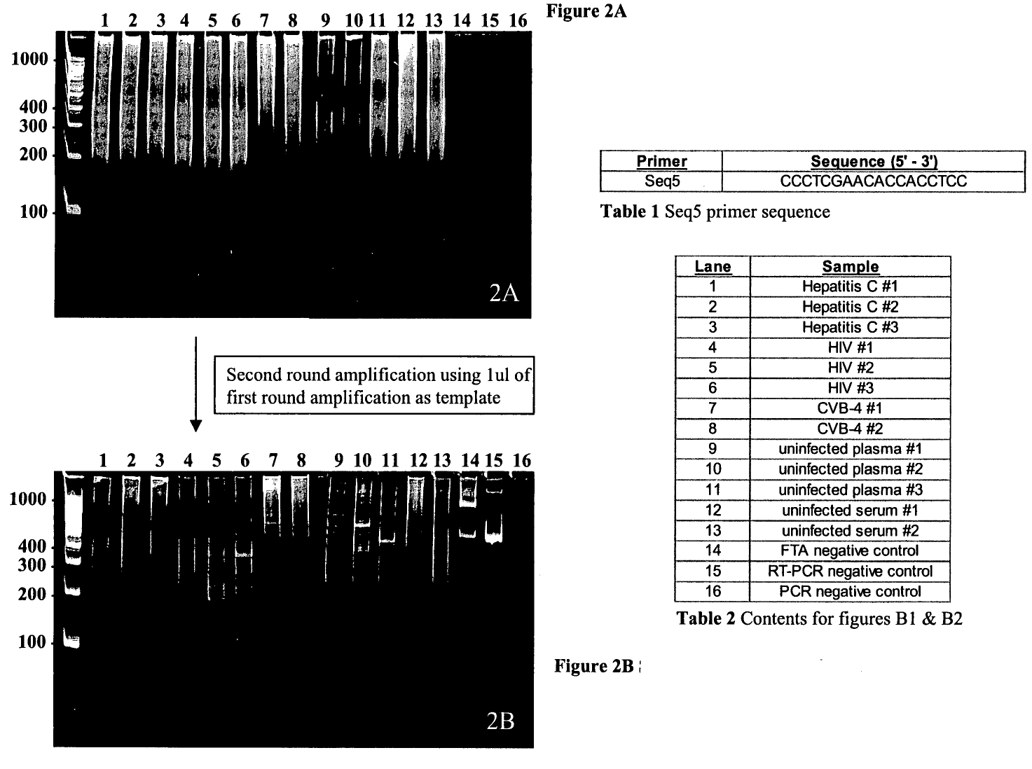

First and second round amplification results were visualized on 7% polyacrylamide gels, which were stained with ethidium bromide and viewed under UV light. Examples of results generated using the procedure are set forth in FIG. 2.

Discussion/Conclusion

The interpretation of the data in FIG. 2 is not governed by any particular banding pattern, but by the visibility and/or frequency of band formation. The frequency of band formation inversely correlates with relative amounts of free nucleic acids in a given sample. All samples (except where specified) were obtained from sera, and thus were expected to contain large amounts of cell-free nucleic acids (template) due to the cell breakdown that occurs during the clotting process (Lee et al., (2001) Transfusion. 41:276-282). Additionally HCV infected sera samples were expected to contain more cell-free nucleic acid due to the cytopathic effect of the virus on hepatocyte cells within the patient. First and second round amplifications generated from HCV and control sera samples resolved as a smooth polydispersed pattern, with a wide range of molecular weight. This pattern is an indicator of high levels of template.

In a healthy individual, samples of plasma contain minimal cell-free nucleic acids, and thus there is limited template for a PCR reaction. Amplification generated from clinical plasma samples was visualized as a set of discrete bands, indicating minimal template.

One important practical aspect of estimation of trace template by SMIPS™ primer amplifications is that it allows template estimation by two totally different principles: the first being the determination of the amounts of DNA by conventional estimation of the amounts of amplification product after a set standard reaction; and the second being the determination of the amounts of template by usage of the loss of molecular diversity during amplification. Loss of molecular diversity can be estimated by a range of methods known to those in the field. Examples of such methods include observation of renaturing rates of products during the process of amplification; observing the ratio of material found in discrete peaks as compared to that found in background smear in a size fractionation of the products; and usage of loss of molecular diversity as a measure of the amount of original template from any given genomic source.

Amplification from the HIV-infected sera samples produced a discrete set of bands, similar to those observed from plasma. This type of pattern is an indicator of limited template and is not unexpected from this type of sample that is expected to be depleted in cells. A part of the life cycle of HIV is to infect cells containing the CD4+ receptor (Kuby, (1994) Immunology 2^{nd} Edition, W. H. Freeman and Company, USA; Collier et al, (1993) Human Virology, Oxford University Press Inc., USA). Thus HIV infects white blood cells and eventually causes cell death. Literature indicates that advanced cases of HIV result in depletion of white blood cells from the blood (Kuby, (1994) Immunology 2nd Edition, W.H. Freeman and Company, USA; Collier et al, (1993) Human Virology, Oxford University Press Inc., USA). The cytopathic effect of HIV on white blood cells results in a significant decrease in nucleated cells in the blood, and thus less cell-free nucleic acid produced during clotting.

Further analysis of the first and second round amplification products via sequencing reveals that the type of nucleic acids amplified also divulges useful information about the disease state. Sequences obtained from HIV sera samples contained an increased number that originated from the mitochondria with respect to the nucleus, in conjunction with a lack of whole cell genomic nucleic acid. Mitochondrial sequences were rarely observed in amplifications from non-HIV subjects. Additionally, sequences generated from HCV infected sera with the non-specific primers specified provided further information about secondary infections, for example sequences were obtained from Pseudomonas aeruginosa (Picot et al, (2001) Microbes & Infection. 3(2): 985-995), Neisseria meningtidis (Parkjill et al, (2000) Nature. 404(6777): 502-506) and Burkholderia cepacia (Mohr et al, (2001) Microbes & Infection. 3(5): 425-435), all of which have been documented as disease-related opportunistic pathogens.

This type of analysis is not limited to infectious disease, but could also be a powerful tool in cancer diagnosis. Published studies on the serum from tumor patients detected tumor DNA (Jahr et al, (2001) Cancer Research. 61: 1695-1665), but this failed to give insight into the size or location of the tumor. Study of the sequence content of mRNA fragments in serum or plasma, because of the expected short half-life of RNA, can be a powerful tool in the detection of the tissue that is dying around the tumor and/or the nature of the tumor itself.

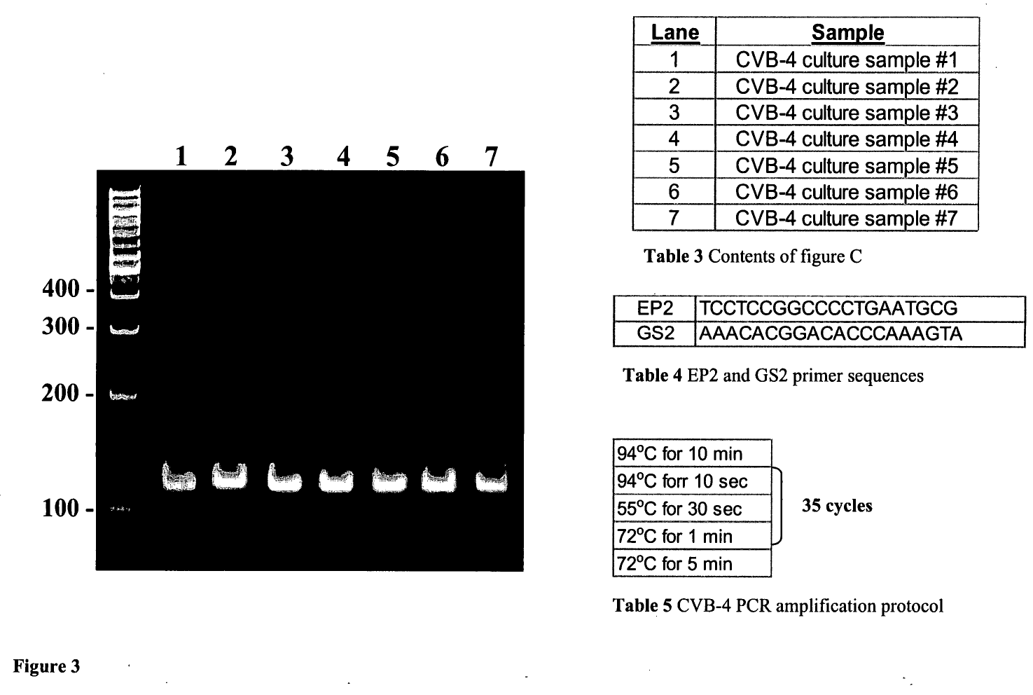

Confirmation of the presence of the infectious agent was also observed with a further pathogen specific amplification. FIG. 3 displays amplification results obtained when a CVB-4 specific amplification was carried out on a range of products (5 μl) from first round PCRs. The pathogen specific reactions generated the 110 bp fragments that indicated the presence of CVB-4. The 110 bp fragment was sequenced and confirmed CVB-4.

The technology described in this patent has novel application to infectious disease diagnostics because, in addition to supplying information about the presence of a particular pathogen, the technology can, on the same blood sample, provide information about the health or disease state of an individual. The technology also has the ability to detect microbial entities directly or indirectly that may not be expected or suspected (i.e. secondary infections by opportunistic pathogens). Additionally the technology also has clear applications to the diagnosis of unknown diseases, or in situations where diagnostic tests are not available or do not exist.

### Example 2

### Detection of DNA Breakage

All blood and bacterial samples used in the study were stored on FTA® paper, however FTA® paper was not essential to generate results, and was a convenient medium for the manipulation of biological samples. 1 mm discs containing blood samples of age one day, 14 days and 1095 days were used, additionally 1 mm disc samples of E. coli of one day old were also used.

All samples were phenol processed prior to amplification or methylene blue photolysis. When used, methylene blue treatment consisted of 5 μl of methylene blue solution (50 μg/ml methylene blue, 2 mM TE, pH 7.5) applied to a 1 mm disc and then exposed to an 8-Watt white fluorescent lamp for a period of 15 minutes at a distance of 6 cm. The intensity of the lamp output at this distance was 3.02 kWm⁻² (Murov, (1973) Handbook of Photochemistry, Marcel Dekker Inc, New York). Methylene blue treated samples were further phenolically processed before amplification.

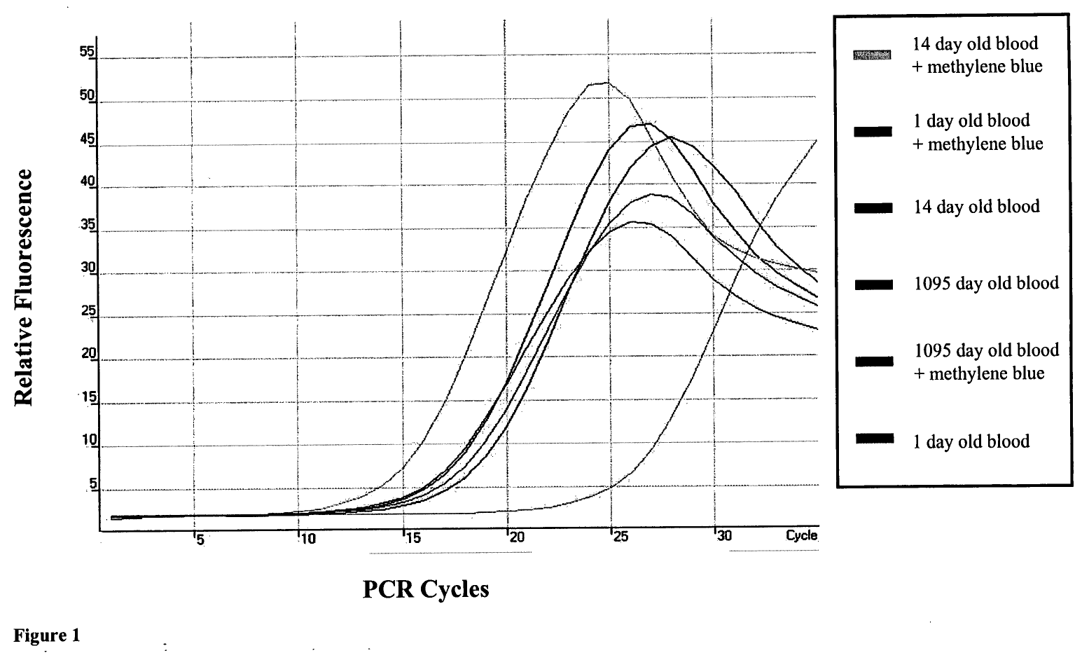

Amplification of the 1 mm samples was conducted using the temperature cycling protocol in Table 2, the primer Seq005 and SYBR® Green (other fluorescent ligands may also be used). Amplification and analysis was carried out on a Rotor-Gene 2000 Real-Time Cycler, with acquisition of fluorescence during the extension phase of PCR using an excitation wavelength of 470 nm and an emission wavelength of 510 nm. Results can be observed in FIG. 1.

Discussion/Conclusion

The data displayed in FIG. 1 shows a large difference between the amplification intensity of one day old whole blood±methylene blue photolytic treatment. The apparent difference in amplification intensity appears to decrease with storage time on the FTA® paper. Literature indicates that methylene blue is a photolytic agent capable of causing single and double strand breaks in DNA (Epe et al, 1989; Hong, 2000; Schneider et al, 1990). The SMIPS amplification process provides a straightforward method for the monitoring of genome integrity and DNA damage.

The observation that the effect of the methylene blue treatment decreased in some sort of relation to storage time on FTA® paper is not unexpected. Evidence from previous studies involved in the investigation of nucleic acid storage on FTA® suggest that variations in temperature storage conditions may possibly induce nucleic acid bond breakage either and/or via DNA conformation changes or mechanical stresses directed from the FTA® paper (Hong, (2000) PhD Thesis, Flinders University of South Australia).

The applications of the methylene-blue-light-induced effects are significant for both commercial and scientific research. It shows the existence of a unique and sensitive method for DNA damage monitoring and detection.

It has been occasionally noted that large templates are strangely bad templates for some PCR primers but this effect has been described in such a way that it is only recognized as an annoying disadvantage of no practical application and it has been described with template specific primers. This discloses the use of conditions that maximize the effect by using non-template specific primers and conditions.

The detection and monitoring of DNA damage, particularly DNA circulating as a result of cell death against a background of normal DNA in white blood cells can be accomplished by the present invention. Examples of monitoring that can include monitoring tissue breakdown and the monitoring of DNA modifications in “at-risk” professionals (e.g. nuclear industry workers after accidents), or the evaluation of the side effects of radiation therapy and chemotherapy on patients. Analysis of a patient's current level of DNA modification (e.g. accumulated mutations or strand breaks in circulating DNA) aids practitioners in accurately diagnosing the correct levels of treatment without risking the patient. Additionally it is desirable and essential for preventative medicine in determining if an individual has a low DNA repair capacity, for example individuals with a family history of breast cancer (Leong et al, (2000) International Journal of Radiation Oncology, Biology, Physics. 48(4): 959-965), and is thus at greater risk in the development of a life threatening malignancy.

### Example 3

### Microbial Population Genetics

DNA Extraction and Amplification: Soil samples were collected from five distinctly different geographical sites. DNA was extracted from each soil sample using the MoBio Soil DNA Extraction kit, (address and contact details) however the kit was a convenience item and other kits and/or methods can be also applied.

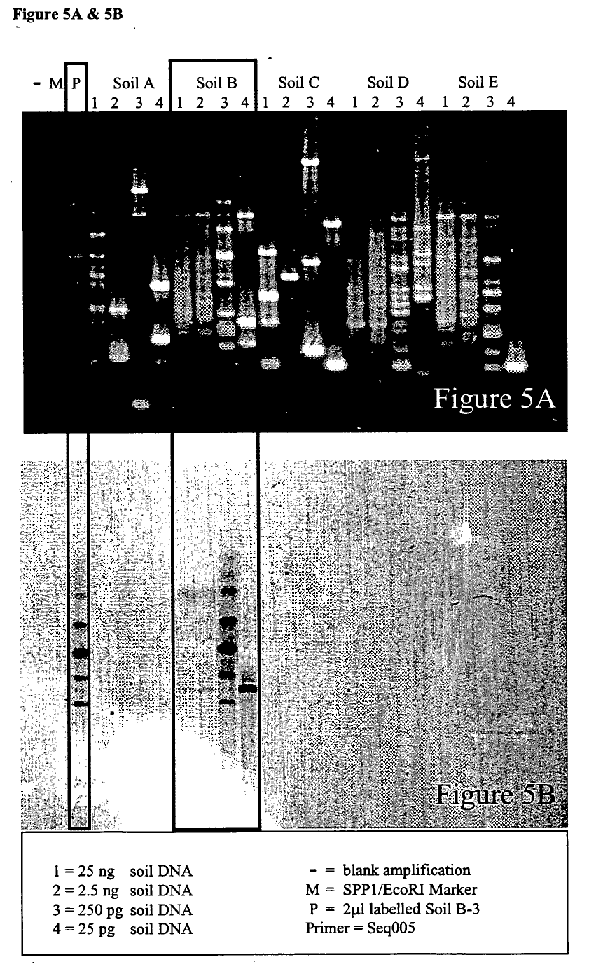

A range of DNA concentrations were used as templates for the first round of PCR, 25 ng, 2.5 ng, 250 pg and 25 pg (represented as lanes 1, 2, 3 & 4 in FIG. 5 respectively). A first round amplification was carried out on each of the DNA concentrations from each of the soil samples using Seq005 as the primer. The PCR protocol was the same as in Table 2. The template for the second round amplification consisted of 5 μl of the first round amplification product. The second round amplification used Seq5 as the primer and the PCR protocol in Table 2.

Probe Labeling: The probe was generated by using 5 μl of the first round PCR product of soil B-3 (see FIG. 5), and re-amplified (using the same primer and protocol) in the presence of 50 μCi S³⁵ dATP.

Southern Blotting: The blotting was performed as described in Sambrook et al, (1989) Molecular Cloning—A Laboratory Manual, 2^{nd} Ed. Cold Spring Harbour Laboratory Press, USA, except the membrane used was ZetaProbe (BioRad).

Probe Hybridization & X-Ray Film Exposure: These methods were performed as described in Sambrook et al, (1989) Molecular Cloning—A Laboratory Manual, 2^{nd} Ed. Cold Spring Harbour Laboratory Press, USA.

Results/Discussion

The amplification patterns observed from the five soil samples are in agreement with previous SMIPS amplification results (e.g. the infectious disease example). Samples containing greater quantities of template generate a larger range of amplification species, and thus molecular-competition sufficient to cause band formation requires more amplification cycles.

The Southern blotting does indicate that amplification species generated from each PCR are unique to the sample of origin, as no cross-reactivity was observed after hybridization (FIG. 5). However cross reactivity was detected between PCR from the same soil sample, as the probe generated from soil B-3 also hybridized to other amplification products from soil B.

The evidence in this example are in accord with the expectation that the SMIPS amplifications generate a “profile” of a highly complex and unknown mixture that is a, representative of a given template mixture without necessarily being a full cross section of a given template mixture. It is a profile that is unique to that sample when compared to an identical amplification generated from other template mixtures, even though portions of the templates may share similar sequences.

These unique sequence-characteristics of the amplification of species between complex template samples provide an opportunity for the adaptation of this technology to areas such as microbial profiling and forensics.

Microbial Profiling: The profile generated from organism has the potential to act as a marker of close relatedness as the closer two organisms are genetically related, the more amplified species they have in common.

Forensics: The example provided in FIG. 5 demonstrates that the microbial flora present in a given soil sample is the distinctive property of a given soil at a given site or region. This observation can be a useful tool in a forensic investigation in which samples exist that contain mixtures of nucleic acid that maybe compared to an original source.

### Example 4

### Quality Control

SMIPS amplifications were carried out on a variety of human DNA concentrations, ranging from 310 ng, 31 ng, 3.1 ng and zero. Amplifications were carried out using four different enzyme suppliers, but all using the Seq005 primer and the PCR protocol in Table 2. The resultant amplification products were visualized on a 1.5% agarose gel and stained with ethidium bromide.

Discussion/Conclusion

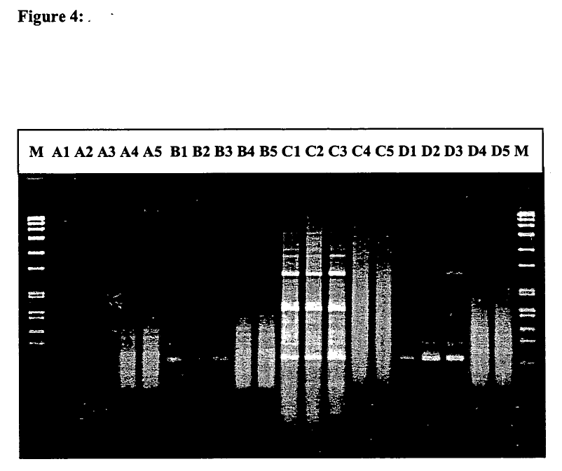

The results displayed in FIG. 4 are an example in the detection of trace levels of nucleic acids, or quality control in commercial DNA polymerase preparations. The presence of traces of contaminating nucleic acids in an enzyme preparation can have a dramatic effect on the outcome of the amplification reaction (e.g. production of false and misleading amplification species). FIG. 4 displays the effect of contaminating nucleic acid on negative controls. In this figure all manufacturers produced a false positive background, with manufacturer A the best, and manufacturer C the worst. The contamination from supplier A appeared to be in the enzyme mix and not the reaction buffer.

Another example in which the detection of unknown and unsuspected nucleic acids is important is in health products are for monitoring water and food quality. All unsuspected nucleic acids are undesirable contaminants of injected materials.

It is essential that healthcare products, particularly those that are used intravenously (e.g. antibiotics, vaccines, other blood related products) do not contain unsuspected nucleic acids, for example, fragments of viral or bacterial genomes. The consequences of modulating an infection with extraneous nucleic acids via the use of contaminated healthcare products are large in terms of personal health and potential litigation. Free nucleic acids are well known to transfer drug resistances and virulence between microorganisms.

Free nucleic acids can be incorporated into and expressed in human cells with the possibility of insertional mutagenesis and/or the expression of strange polypeptides with unknown consequences.

The screening of consumable materials such as water and food for unknown nucleic acids is also of prime importance, as the contaminating nucleic acids can be an early indicator of bacterial or viral contamination. Consumption of contaminated food or water is a serious health risk, routine monitoring of foods and water with the SMIPS based technology can significantly decrease the incidences of infection and disease, decreasing the risk to the consumer.

### Example 5

The program provided is an example that can be utilized to create suitable primers. The program must be able to create a sequence rich in C but low in G. The program can run on any common operating system.

The example program, below, is for 18 bases long and is as follows:

′Prog1 - a program in Borland's Turbobasic. Black text is comment and red text is
compile-able code.
′This makes primers according to a set of rules.
′ The products of this family of primers are to be 18 base pairs long.
′The products are to start with a fixed sequence at the 3′ end of (5′CCTCC3′)
′The products are to then have 13 additional bases added with the following
characteristics: 55% C (7C), 30% A (4A) and 15% G and T (1G and 1T)
′Effective self complementarity is avoided by choice of high C with only 1G .
′There should be no very long runs of homopolymer. (Runs of >3 of any base will
cause a candidate sequence to be rejected and runs of A longer than 2 are also
rejected.)
′
′START CODE -start by declaring the array stores.

DIM PRIM$(21)
DIM R$(400)

′Declare the base content as an unrandomised sequence to be used in the ,e.g., 13
positions of the 5′ end. Place them in the string store, WD$, which will act as the menu
of bases to be scrambled.

WD$=″CCCCCCCAAAAGT″

′Declare the name of the output file from the keyboard and set randomiser

PRINT ″INPUT THE NAME OF THE OUTPUT FILE FOR THE NEW
SEQUENCES.
PRINT ″with no more than six characters in the name.”

′At the same time, initialise the random generator that Borland provides in their
software.
′the human delay in inputting the file name allows the internal clock to generate a highly
variable seed.

PRINT ″INPUT THE DESIRED OUTPUT FILE NAME″:INPUT NM$
RANDOMIZE TIMER

′Clear the screen of the computer

CLS:PRINT ″ ″

′Begin cycles of producing candidate sequences. Clear the main counter POI% and

POI%=0
FOR R%= 1 TO 2000
POI%=POI%+1:IF POI%=>398 THEN GOTO YNDUPFINAL
PRINT ″CYCLE NUMBER-″;R%

′ Begin a cycle by fixing the first five bases from the 3′ end of the primer, in the
string array store PRIM$(1) where PRIM$(1) is the 3′ base.

PRIM$(1)=″C″
PRIM$(2)=″C″
PRIM$(3)=″T″
PRIM$(4)=″C″
PRIM$(5)=″C″

′Now fill the rest of the primer/oligo in 13 operations filling oligo places 5 TO 18
randomly using a list of numbers from 1 to 13 and then scramble them.
′first, fill a little array with ordered numbers.

FOR J%=1 TO 13:NUM%(J%)=J%:NEXT J%

′Now scramble (disorder) these numbers in the little array.

FOR JJ%=1 T0 13
AGIN:
SS$=INKEY$:IF SS$<>″″ THEN GOTO YNDUPFINAL:′this line of code is only to
allow an overriding termination from the keyboard

′call for a random number from 0 to 14 (but reject it if it is not between 1 and 13
inclusive).

CALL RAN:IF NU%<1 OR NU%>13 THEN GOTO AGIN

′use the random number in a scramble.

IHOLD%=NUM%(JJ%):NUM%(JJ%)=NUM%(NU %):NUM%(NU%)=IHOLD%
NEXT JJ%

′Now use the small list of scrambled numbers to pick out the bases from the
menu of bases in WD$ and place them from the end of the 3′ zone to the end of
the 5′ zone with operations proceeding in the 3′ to 5′ direction.

FOR J%=1 TO 13
PRIM$(J%+5)=MID$(WD$,NUM%(J%),1)
NEXT J%

′now write the candidate oligo's sequence in the reverse (the conventional) direction in
the array store R$( ) while checking for homopolymer runs.

FOR J%=18 T0 1 STEP -1
R$(POI%)=R$(POI%)+PRIM$(J%)
NEXT J%

′Now look for any forbidden runs of any homopolymers.

IKILL %=0:IKILL2%=0:IKILL3%=0:IKILL4%=0
FOR J%=1 TO 17
C$=MID$(R$(POI%),J%,1):D$=MID$(R$(POI %),(J%+1),D
IF C$=D$ THEN IKILL%=IKILL%+1:IF IKILL%>2 THEN IKILL2%=1
IF C$<>D$ THEN IKILL%=0

′The next lines are AAA forbidders.

IF C$=D$ AND D$=″A″ THEN IKILL3%=IKILL3%+1:IF IKILL3%>1 THEN
IKILL4%=1
IF C$<>D$ THEN IKILL3%=0
NEXT J%

′The next lines execute the rejections on either of these two grounds.

IF IKILL2%>0 THEN R$(POI %)=″″:POI%=POI%-1:GOTO ENDOFRR:′reject a
candidate due to homopolymer content.
IF IKILL4%>0 THEN R$(POI %)=″″:POI%=POI%-1:GOTO ENDOFRR:′reject a
candidate due to AAA homopolymer content.

′Now scan each candidate oligo against all those already listed and reject any
sequences already found once.

FOR K%=1 TO (POI%-1)
IF K%=1 AND POI%=1 THEN GOTO SKIPIT
IF R$(K%)=R$(POI%) THEN ICOU%=ICOU %+1:′ noting the number of
candidates rejected for being duplicates before actually rejecting them in the next
line.
IF R$(K%)=R$(POI%) THEN R$(POI %)=″″:POI%=POI%-1:GOTO ENDOFRR
SKIPIT:
NEXT K%
PRINT R$(POI%)
ENDOFRR:
IF POI%<0 THEN POI%=0
SS$=INKEY$:IF SS$<>″″ THEN GOTO YNDUPFINAL:′(another line to allow an
overriding termination from the keyboard.)
NEXT R%
YNDUPFINAL:

′Open the disc to output the list of acceptable sequences and output them..

OPEN NM$ FOR OUTPUT AS #2
PRINT#2, ″The number of identicals observed and rejected in this run =
″;ICOU%
PRINT#2, ″The number of sequences to print-″; POI%
FOR FIN%=1 TO POI%
PRINT#2,R$(FIN%)
NEXT FIN%
PRINT#2,″end″
CLOSE#2
PRINT “Your output is in the file you called- “;NM$
PRINT ”NOW TOUCH A LETTER ON THE KEY BOARD TO END UP THIS
PROGRAM.″
PAUSEND:
SS$=INKEY$:IF SS$=″″ THEN GOTO PAUSEND
END

′****************
′This small subroutine called RAN simply generates random numbers between 0
and 14 and delivers them to NU% for return to the main program.

SUB RAN
SHARED NU%
LOCAL ULIM,LLIM
LOCAL NU,DF,X
ULIM=14:LLIM=0
DF=ULIM-LLIM
GETANUTHER:
X=RND(5)
NU=(X*DF)+LLIM:NU%=NU:IF NU %<LLIM OR NU%>ULIM THEN GOTO
GETANUTHER
END SUB

′*****************************

### Example 6

Analysis of Soil Microbial Population Genetics—an Empirical Measure of the Similarity of Two Extremely Complex Mixtures of Sequences.

Table 3 supplies a list of SMIPS primers that have already been tested. Other “equivalent” primers can be formed and can function in the manner described herein. The primer seq005 was the parent primer of the primers listed in Table 3. Presently primer seq005 is the most successful (in terms of range and rate of sequences amplified). All other primers were designed from seq005.

FIG. 9 represents an experiment in which a single SMIPS primer seq005 was used to SMIPS-amplify a range of soil samples from different locations, human DNA was also SMIPS-amplified as an unrelated control. The product of each amplification was a mixture of sequences. In other words, there were no fixed number of sequences, but the numbers of sequences ranged from about 8 to 12 major species along with a number of minor species.

The products of the SMIPS amplifications were immobilized onto a DNA array as one spot per mixture and then all spots were probed with the amplification product from soil A that had been labeled with fluorescent nucleotides. The data represents levels of relative fluorescence, which is a measure of the DNA hybridization of each labeled amplification product to the array elements of unlabeled amplification products. The results show that the amplification products generated by a SMIPS primer from a given soil sample (soil B) share genetic information between the soil being tested (the +ve control set at 100%) and soil SHR, a different soil, to a degree of approximately 20%. Other soils show much less sequence sharing.

Although the measure of similarity in the above example is accomplished by labeling the product with fluorescent nucleotides and then studying the cross-hybridization present on an array of other, unlabeled products, a highly related comparison can be made by a non-array technique using a terminal restriction fragment technique with fluorescent primers and a mixture of restriction endonucleases and observing the sizes of the fluorescently labeled fragments derived from the many, for example 4 to 12, sequences within the amplification product. The 4 to 12 sequences with labeled primer would be expected to deliver approximately 8 to 24 terminal restriction fragments. Terminal restriction fragments are known as a crude method of sequence analysis and the preferred method is a DNA array.

Amplification of the Human Genome for an Empirical Measure of the Simplicity of Any One Mixture of Sequences and Demonstration of the Difference Between Equivalents and Non-Equivalents

FIG. 8 represents an experiment in which a variety of SMIPS primer equivalents and some non-equivalents were used to amplify sequences from a human genomic template. The amplification products were analyzed for cross-reactions. The analysis was carried out using a DNA array, which had been loaded with the amplification products generated by the range of primers shown on the x axis of FIG. 8 (see table 3 for the sequences of primers) from human DNA.

The resultant array was probed with the amplification products generated by primer seq005. The data represents levels of relative fluorescence, which is a measure of DNA hybridization or cross-reactivity (e.g. the higher the fluorescence the higher the cross-reactivity). Analysis of the results along with the use of antiseq005 as a standard “non-equivalent” primer indicated that the majority of primers, to the left of antiseq005 on the x-axis of FIG. 8 were performing as equivalents to different degrees. There is a large amount of simplicity in the sequence mixture as shown by the fact that the specific, non-equivalent, antiseq005 was cross-reacting with seq005 at almost 40%. All primer equivalents products were cross-reacting 5% above antiseq005 (antiseq005, in theory, is a non-equivalent). Such a large amount of simplicity is expected from the human genome, which is known to contain a large amount of repetition.

The invention has been described in an illustrative manner, and it is to be understood that the terminology that has been used is intended to be in the nature of words of description rather than of limitation.

Obviously, many modifications and variations of the present invention are possible in light of the above teachings. It is, therefore, to be understood that within the scope of the appended claims, the invention may be practiced otherwise than as specifically described.

What is claimed is:

1. A method of amplifying genetic material for profiling or cloning by selecting at least one sequence from a population of sequences for amplifying genetic material and selecting for properties of the sequence found between primer binding sites.

2. The method according to claim 1, wherein said selecting step includes selecting for inter-primer binding site folding.

3. The method according to claim 1, wherein said selecting step includes selecting for interactions between primer sequence termini and sequences found therebetween.

4. The method according to claim 1, wherein said selecting step includes selecting for properties of the sequence that facilitate extension during amplification.

5. The method according to claim 1, wherein said selecting step includes selecting for sequences capable of modifying amplification efficiency.

6. The method according to claim 1, wherein said selecting step includes selecting using a structurally mediated interprimer selectivity (SMIPS) primer selected from the primers in Table 3.

7. The method according to claim 1, wherein said selecting step includes selecting using a primer that is an equivalent of the SMIPS primers listed in Table 3.

8. A method of profiling genetic material by:
selecting at least one sequence from a population of sequences for profiling the genetic material, by selecting for properties of the sequence found between primer binding sites;
amplifying the selected sequences; and
creating a profile from the amplified sequences.

9. A method of sequencing amplified sequences using amplification primers with a 3′ extension of at least 2 nucleic acids.

10. The method according to claim 9, wherein said sequencing step includes analyzing the sequences through a software algorithm that aligns sequences against a known database of sequences.

11. The method according to claim 9, further including identifying confounding or ambiguous sequences by alignment of small regions of the sequence against a known database of sequences and combining the overall result of the alignment of the small regions using a software algorithm.

12. The method according to claim 11, further including adding the result of the software algorithm sequence analysis to a known database as reference.

13. The method according to claim 1, wherein said selecting step includes selecting sequences from the environment.

14. The method according to claim 1, wherein said amplifying step includes amplifying sequences according to tertiary structure of the sequences during annealing and amplification stages.

15. The method according to claim 1, wherein said amplifying step includes modifying amplification conditions to alter the sequences being selected for.

16. The method according to claim 1, further including analyzing the profile.

17. The method according to claim 16, wherein said analyzing step includes analyzing the profile using a technology selected from the group consisting essentially of DNA arrays, DNA chips, and hybridization technology.

18. A device for performing the method according to claim 1, said device comprising;
a robot;
DNA separating and observing means for separating and observing, said robot controls said DNA separating and observing means.

19. The device according to claim 18, wherein said DNA separating and observing means is selected from the group consisting essentially of a capillary electrophoresis machine, an HPLC device, Sanger sequencing fluorescent primer chain terminating technology, and mass spectroscopic technology.

20. A kit for performing the method of claim 1, said kit comprising:
primer sequences; and
a device for amplifying genetic material.

21. A method for amplifying unknown genetic material in a sample by, amplifying the genetic material with sufficient extension times to create at least a 200 bp extension of the material thereby enabling amplification of the unknown genetic material.

22. The method according to claim 21, wherein said amplifying step includes adding SMIPS primer to a smaller amount of the genetic material.

23. The method according to claim 21, wherein said amplifying step includes:
annealing a non-target sequence specific primer for at least one cycle at very low specificity and then repeatedly amplifying the genetic material with a higher specificity wherein there is no amplification from the original template, thereby making selectivity dependent upon the properties of the sequence found between the amplified sections.

24. The method according to claim 9, for use in recording and cataloguing unidentified organisms.

25. A method for amplifying unknown genetic material in a sample by:
collecting the sample;
maintaining the sample in storage media;
purifying the sample;
reverse transcribing genetic material of the sample;
performing PCR on the genetic material; and
analyzing the results of the PCR.

26. A computer program for creating SMIPS primers for use in the method according to claim 1.
